(19) United States
(12) Patent Application Publication
(10) Pub. No.: US 2020/0013504 A1
(43) Pub. Date: Jan. 9, 2020

(54) Patient Behavior Monitoring and Modification System

(71) Applicant: Neurostim Technologies, Inc., Waltham, MA (US)

(72) Inventors: Hoo-min D. TOONG, Cambridge, MA (US); William C. ALTMANN, Austin, TX (US)

(21) Appl. No.: 16/571,878

(22) Filed: Sep. 16, 2019

Related U.S. Application Data
(63) Continuation-in-part of application No. 16/117,133, filed on Aug. 30, 2018.
(60) Provisional application No. 62/731,486, filed on Sep. 14, 2018.
(60) Provisional application No. 62/576,755, filed on Oct. 25, 2017.
(60) Provisional application No. 62/576,839, filed on Oct. 25, 2017.

(51) Int. Cl. G16H 40/63; G16H 40/67

(52) U.S. Cl. CPC G16H 40/63; G16H 40/67

(57) ABSTRACT

Embodiments monitor a patient by generating current behavior data for the patient and comparing the current behavior data to historical behavior data for the patient. Based on the comparing, embodiments determine one or more first anomalies of the current behavior data and in response to the determined first anomalies, modify the behavior of the patient.

### CROSS REFERENCE TO RELATED APPLICATIONS

This application claims priority to U.S. Provisional Patent Application Ser. No. 62/731,486 filed on Sep. 14, 2018, and is a continuation-in-part of U.S. patent application Ser. No. 16/117,133, filed on Aug. 30, 2018, which claims priority to U.S. Provisional Patent Application Ser. No. 62/576,839, filed on Oct. 25, 2017, and to U.S. Provisional Patent Application Ser. No. 62/576,755, filed on Oct. 25, 2017. The disclosure of each of these applications is hereby incorporated by reference.

### FIELD

One embodiment is directed generally to a computer system, and in particular a computer system for monitoring and modifying the behavior of a patient.

### BACKGROUND INFORMATION

Patient monitoring systems have improved the ability of caregivers in the critical role of overseeing the care of patients by providing a reference framework of both requirements and status of patients. Patient checks, treatments and care, which has been routinely monitored by caregivers, and which has become more complex and difficult with increasing patient loads and resources, has been augmented with the introduction of computers to track, remind and record much of the required and given patient care.

### BRIEF DESCRIPTION OF THE DRAWINGS

FIG. 1 illustrates a monitoring system in accordance with example inventions.

FIG. 2 illustrates devices for monitoring the use of a bathroom by a user/patient 220 in accordance with example inventions.

FIG. 3 is a block diagram of a computer server/system in accordance with examples of the present invention.

FIG. 4 is a flow diagram of the functionality of one or more of the elements FIGS. 1-3 for behavior monitoring and modification of a patient as disclosed herein.

FIG. 5 illustrates an example smart diaper and data collection system.

FIG. 6 illustrates various example communication signals of the data collection system of FIG. 5.

FIG. 7 illustrates an example environment for use with the system of FIG. 5.

FIG. 8 further illustrates an example environment for use with the system of FIG. 5.

FIG. 9 illustrate another example smart diaper.

FIG. 10 is a block diagram of an example base station device in communication with a monitoring device.

FIG. 11 illustrates an example smart diaper and data collection system.

FIG. 12 illustrates a standing user with a diaper pulled up to the waist position, with monitoring device positioned relative to base station.

FIG. 13 illustrates four positions of the monitoring device relative to the base station.

FIG. 14 is a flow diagram of the functionality of a base station and/or host device when determining smart diaper based data and analytics.

### DETAILED DESCRIPTION

In general, “patients” or “care receivers” refers to individuals under the care of a “caregiver”. Patients generally require assistance due to their own limitations in self-supported care. However, caregivers who are physically distant from their patients are inhibited in their ability to support the patient's needs in their living space. Further, caregivers who are co-located with the patient need the assistance of alerts and notifications about events which require attention, while also mediating urgency of care.

Caregiving problems that need to be solved include: (1) privacy issues resulting from constant caregiving duties; (2) sleep issues because of interrupted/modified sleep cycles; (3) isolation for the caregiver because of lack of support networks and shared responsibilities; (4) lack of control because of the constant problems/crises that need to be addressed; and (5) time/attention consuming tasks such as a patient's incontinence.

In order to solve some or all of the above problems, inventions disclosed herein allow patients to administer self-care using the diagnostic outputs of an analysis system, such as monitoring changes in their activities in the bathroom, and analysis of biometric data collected and then processed with larger database information to detect and report changes.

The system of example inventions for monitoring and modifying the behavior of patients includes surveillance and activity monitoring devices which are capable of image analysis in real time, using machine learning and artificial intelligence (“AI”) techniques. Example systems further include wearable devices and health related Internet of Things (“IoT”) devices that are either worn by the individual or are accessible in the individual's environment. Data from such devices can be of a real-time nature or can be event based.

Example systems further include social media in all its varied forms and digital assistant devices, employing voice recognition, gesture recognition, and the like. The digital devices can use AI techniques along with extensive databases to provide natural voice interaction and can be valuable complements to the other described technologies. Example systems further include predictive capabilities, using large databases and learning techniques.

In general, a caregiver carries a responsibility to monitor and attend to the needs of a patient. These responsibilities are time consuming, draining, and shown to isolate the caregiver as a result of this role and its demands. The caregiver may be a professional in an institutional care giving facility, or in a private home, or can be a child, parent or relative of the patient.

Example systems, using some or all of the above technology, each provide data to an analysis system that is used in combination so that the analysis system monitors the activities of the patient, and then calculates patterns of normal activities from historical data of the patient or by data mining of population data to distinguish from “normal” activities. Deviations or anomalies from the normal pattern cause example inventions to analyze other possible contributing factors, such as the patient's calendar events, change of diet, change of medication, etc. Based on recognition of abnormal activities or anomalies, notifications are initiated to one or more of the patient, the caregiver, and care providers at the patient's location.

In examples, the frequency of data collection from surveillance and IoT devices is modified by the analysis system based on deductions from the gathered data in real time. If the analysis system detects an abnormal situation (i.e., anomaly), it acts to solicit up-to-date data from a variety of its network of devices.

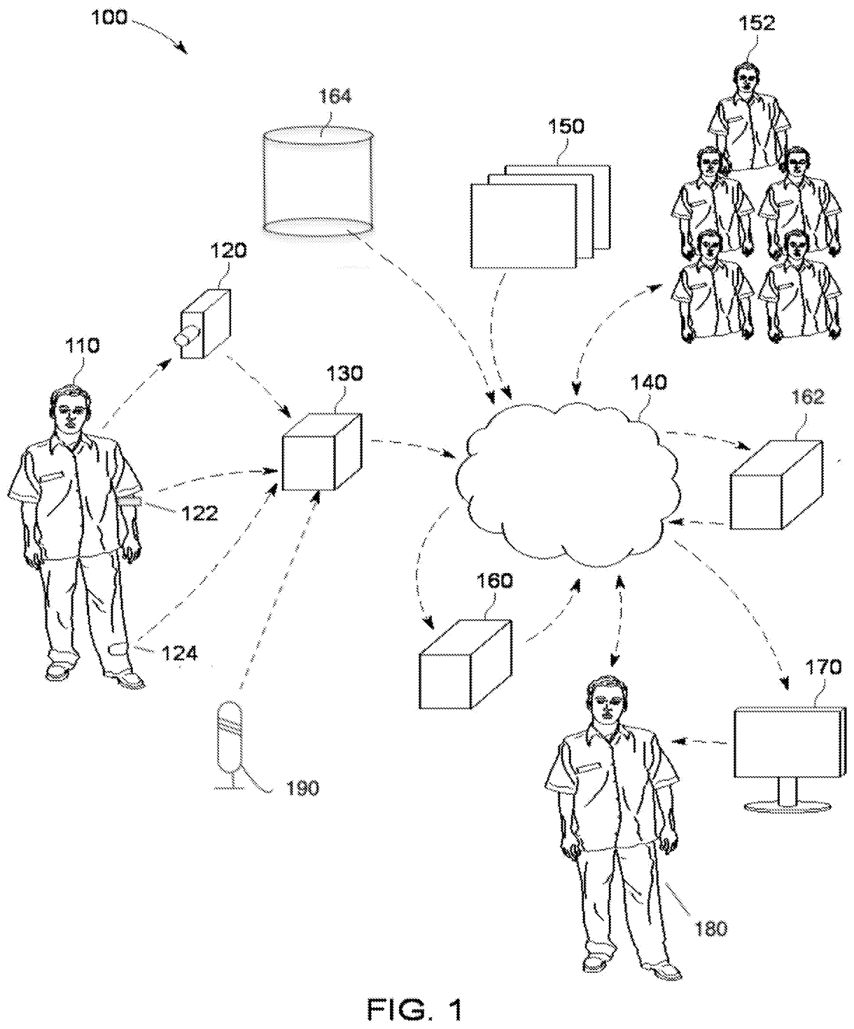

FIG. 1 illustrates a monitoring system 100 in accordance with example inventions. Monitoring system 100, in conjunction with a patient 110, a caregiver 180, and social media participants 152, includes a monitoring device 120, a fitness device 122, a smart patch 124, a controller 130, a cloud service 140, one or more social media sites 150, an analysis system 160, a server 162, a health database 164, a reporting system 170 and a digital assistant 190.

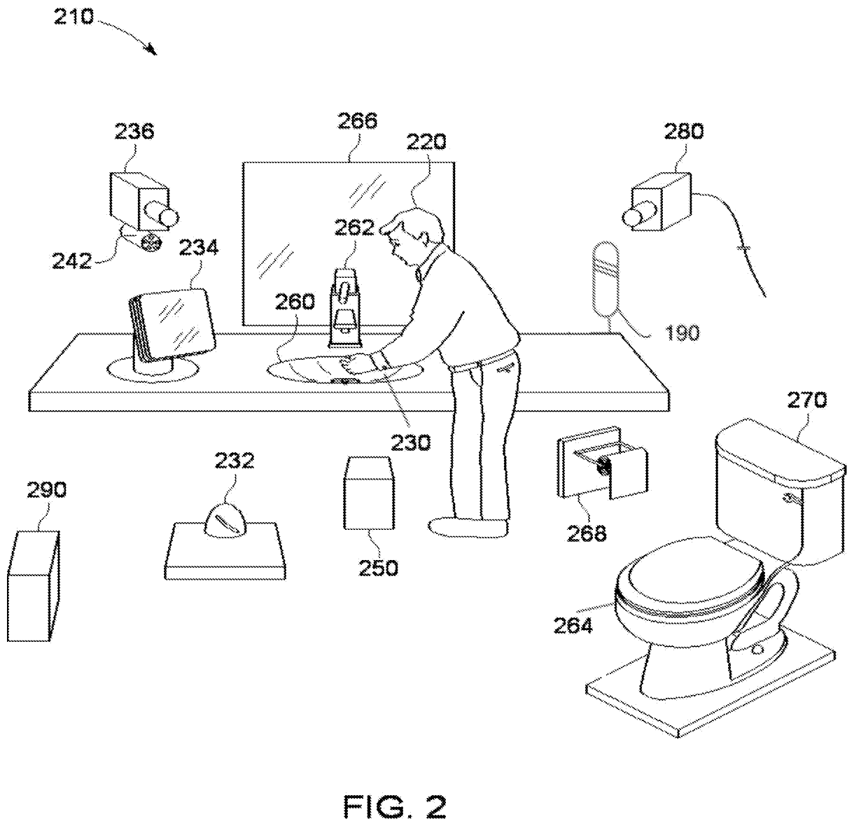

FIG. 2 illustrates devices for monitoring the use of a bathroom 210 by a user/patient 220 in accordance with example inventions. The devices include a wireless fitness device 230, a weight monitoring device 232, a monitoring mirror 234, a monitoring camera 236, an audio input device 242, a wireless hub 250, a sink 260, a faucet 262, a toilet 264, a mirror 266, a toilet tissue dispenser 268, one or more wireless accessories 270, such as a water usage meter or smart toothbrush, one or more non-wireless accessory 280, such as a motion-detecting camera, and a controller 290.

Monitoring devices 120, wireless accessories 270, and similar wireless devices can communicate through Bluetooth, Wi-Fi, or similar means. The wired devices can communicate through USB, optical channels, or other non-RF means. Analysis system 160 can include software, hardware, or both. Social media systems may include blogs, web pages, chat rooms, e-mails, notifications, subscription-based services, membership-based services, etc.

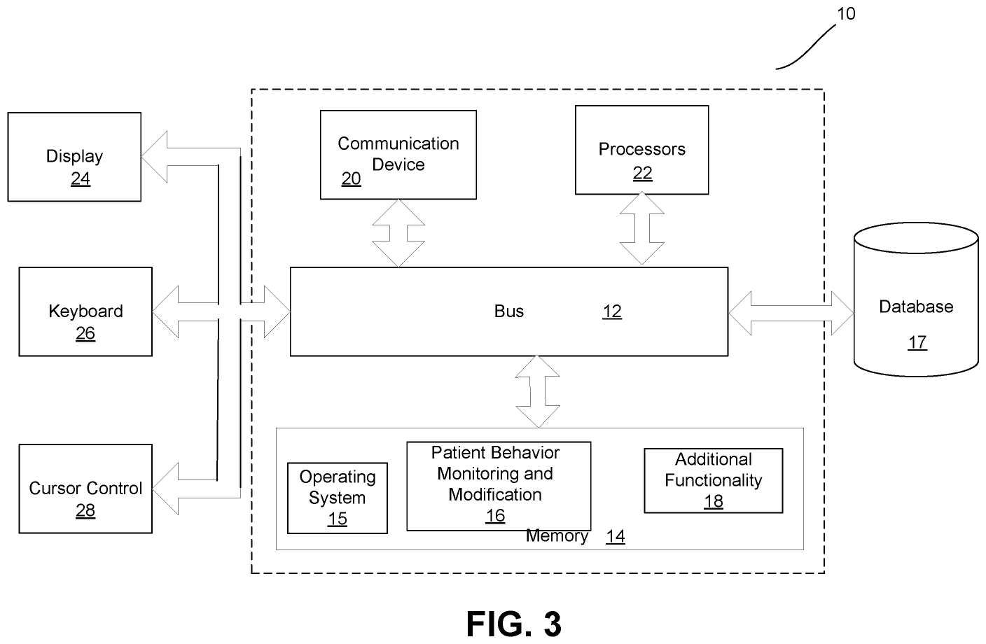

FIG. 3 is a block diagram of a computer server/system 10 in accordance with examples of the present invention. Although shown as a single system, the functionality of system 10 can be implemented as a distributed system. Further, the functionality disclosed herein can be implemented on separate servers or devices that may be coupled together over a network. Further, one or more components of system 10 may not be included. For example, for functionality of a server, system 10 may need to include a processor and memory, but may not include one or more of the other components shown in FIG. 3, such as a keyboard or display. All or portions of system 10 may be used to implement any or all of the components shown in FIGS. 1 and 2 in some examples.

System 10 includes a bus 12 or other communication mechanism for communicating information, and a processor 22 coupled to bus 12 for processing information. Processor 22 may be any type of general or specific purpose processor. System 10 further includes a memory 14 for storing information and instructions to be executed by processor 22. Memory 14 can be comprised of any combination of random access memory (“RAM”), read only memory (“ROM”), static storage such as a magnetic or optical disk, or any other type of computer readable media. System 10 further includes a communication device 20, such as a network interface card, to provide access to a network. Therefore, a user may interface with system 10 directly, or remotely through a network, or any other method.

Computer readable media may be any available media that can be accessed by processor 22 and includes both volatile and nonvolatile media, removable and non-removable media, and communication media. Communication media may include computer readable instructions, data structures, program modules, or other data in a modulated data signal such as a carrier wave or other transport mechanism, and includes any information delivery media.

Processor 22 is further coupled via bus 12 to a display 24, such as a Liquid Crystal Display (“LCD”). A keyboard 26 and a cursor control device 28, such as a computer mouse, are further coupled to bus 12 to enable a user to interface with system 10.

In one embodiment, memory 14 stores software modules that provide functionality when executed by processor 22. The modules include an operating system 15 that provides operating system functionality for system 10. The modules further include a patient behavior monitoring and modification module 16 that provides patient behavior monitoring and modification functionality, and all other functionality disclosed herein. System 10 can be part of a larger system. Therefore, system 10 can include one or more additional functional modules 18 to include the additional functionality. A database 17 is coupled to bus 12 to provide centralized storage for modules 16 and 18. In one embodiment, database 17 is a relational database management system (“RDBMS”) that can use Structured Query Language (“SQL”) to manage the stored data.

Surveillance And Activity Monitoring

In many cases, the distance between caregiver 180 and patient 110 makes the caregiving role more difficult and time consuming, as caregiver 180 needs to rely on periodic information that is received, or by periodic physical visits to patient 110 across that distance. The distance between caregiver 180 and patient 110 can be as small as in another part of the house, or as large as across the nation or even across continents. Modern communications technology such as the Internet, make such a caregiver/patient relationship possible. Distance can cause caregiver 180 to feel overwhelmed by the time commitment to patient 110, and cause a loss of sleep worrying about the condition of patient 110 in a remote location. Continuous information can be available to caregiver 180 through the use of camera 236 and microphone 242 in the patient's environment, sending the data streams to caregiver 180. Surveillance devices can, through the use of artificial intelligence and machine learning, distinguish between patients and other individuals who can use the facilities.

Networked Devices

A variety of devices can monitor the patient's health data (i.e., biometric data), or the corresponding patient environment, such as temperature. All of these devices can be connected, through wired or wireless means, so that data and commands can flow between devices, or between devices and a controller. The aggregate data can be sent to caregiver 180 for ongoing monitoring of the patient's condition and activities. This aggregate data can be analyzed by software to reduce the time needed from caregiver 180 to continue the same level of care to patient 110. The analysis may be done by dedicated or shared computer resources, and may be done by larger analysis systems which analyze data from a population of inputs.

Mesh networks and body area networks may be used to collect and aggregate data from patient 110. For example, a mesh network in a home or caregiving facility may provide peer-to-peer connectivity among surveillance, IoT and other devices. These devices may interact directly as patient 110 moves about in the facility or as the patient's state changes. For example, patient 110 waking from sleep, or falling asleep, may be detected by one or more IoT devices, which may then notify peer devices, these peer devices then slowing or stopping their data collection while patient 110 sleeps, or restarting their data collection as patient 110 wakes. A body area network may perform the same optimizations, aggregating the data on a per-user basis, and sending it to the connected system to notify caregiver 180.

Social Media

Internet websites, including social media websites 150, can be available to caregiver 180, to provide information and supportive dialog on topics related to caregiving to patient 110, and to the health of the caregiver 180. The inclusive nature of these social media resources reduces the caregiver's sense of isolation. The employment of social media sites can be the result of data analysis of data from surveillance or IoT devices. It can also assist caregiver 180 to recognize unusual or problematic actions of patient 110.

Digital Assistants

Digital assistants in proximity to the patient 110 can be used to solicit input from patient 110, under direction from the analysis system, or from caregiver 180, or from other care providers at the patient's location. They can employ natural language interfaces and even employ the actual speech signals that mimic caregiver 180, thus providing a high degree of trust and comfort to patient 110. Speech patterns can be used to detect a patient's state of mind and indicate changes in health, through diagnostic use of the analysis system.

Large Databases and Learning Techniques

Data from caregiver 180 can be combined with data from a larger population through the use of a health database 17, thereby improving the diagnostic abilities of the analysis system based on correlation of the patient's data to data in the database. Recommended actions can be prioritized and sent to caregiver 180 and patient 110, to take action with patient 110, or sent to patient 110 for awareness and for them to take their own action. This analysis can be performed with the assistance of machine learning and artificial intelligence techniques.

Invention examples that incorporate one or more of the above technologies can be predictive in nature based upon machine learning and deep learning techniques operating upon historical data of patient 110 and/or general population data to detect, analyze and predict health issues.

Use Cases

Surveillance camera 236 in bathroom 210 in examples can be used with motion detection software, artificial intelligence software, machine learning software, etc., to detect when patient 110 goes into or comes out of bathroom 210, and the activities they perform while in bathroom 210. For example, showering is an important patient activity that can be monitored for normal and abnormal activity such as falls or cries for assistance. In examples, software delivers a report to caregiver 180 on the regularity of the patient's use of toilet 264. Deep learning techniques can make predictive assessments of specific events, such as a probability of fall in the shower based upon ongoing progressive patterns of behavior.

Wireless connections from IoT devices such as a smart toothbrush, and a smart soap dispenser can transfer data from these devices to a controller, such that software can analyze how patient 110 is adequately using or not using the toothbrush to maintain dental health, or using soap to maintain cleanliness. Software can deliver a report to caregiver 180 on the adequacy of the patient's self-care.

Caregiver 180 can subscribe to or otherwise participate in social media groups, such as groups for specific medical conditions of the patient's health, or groups for discussing and suggesting solutions for the issues felt by caregiver 180 such as loss of sleep, interruptions to sleep, time spent in traveling to and from the client, etc.

Patient 110 can be informed through digital assistant device 190 that analysis system 160 and observation data have deduced an abnormal condition or anomaly. Analysis system 160 sends a request to digital assistant 190, which then queries patient 110 through audio to request a confirmation from patient 110 that the conditions are normal, or are abnormal, such as distinguishing between patient 110 intentionally sitting on toilet 264 for a long time, versus patient 110 needing assistance after being unable to rise from toilet 264. If patient 110 responds to digital assistant 190 that no assistance is needed, then analysis system 160 realizes that the condition is normal, and no request for assistance is sent to caregiver 180. If patient 110 responds that help is needed, then a request is sent to caregiver 180 or other resource that help is needed.

In each of the above examples, the caregiver's time spent in delivering care to patient 110 can be reduced, and the quality of life of caregiver 180 can be improved. In addition, the caregiver's time spent in analyzing the behavior of patient 110, in order to make decisions on what care to deliver, can be reduced through the systematic analysis of the data and distinguishing normal from abnormal activity.

In example inventions, the surveillance devices and IoT devices can work in combination to deduce the patient's actions through software analysis, such as the simple tracking of behavior by noticing the use of toilet 264 followed by use of sink 260 with soap and water. Additional software analysis can incorporate aspects of machine learning and deep learning to understand the deeper implications of abnormal behavior against prior patterns or patterns from a larger population of patients. Rather than personally monitoring the patient's activities with toilet, soap and water, caregiver 180 can be notified only when the patient's activities are outside the normal pattern.

Another example is the critical issue of medication compliance. IoT devices can work in combination with online resources such as the patient's Electronic Health Record (“EHR”). IoT sensors can be included in the patient's medicine bottles, as well as on the medicine cabinet door. The analysis system can deduce that patient 110 has visited bathroom 210 in the normal pattern, but has not opened the medicine cabinet door, or has not opened one or more of their medicine containers for a period longer than the prescription period defined in the EHR. The analysis triggers a notification to caregiver 180 to visit patient 110. If the specific medication warrants a critical situation, the analysis system can send a message to on-site care providers at the patient's location.

In examples, IoT devices can also be placed outside of bathroom 220 to monitor medicine usage (e.g., in the kitchen). Other types of medical compliance can be monitored, such as use of physical therapy devices for the prescribed times. Physical activities which violate the limitations set by the patient's doctor may also be monitored, such as walking without crutches, failure to wear a prescribed therapeutic device, failure to exercise as prescribed, etc.

The privacy of patient 110 can be improved over continual in-person time with caregiver 180 by using surveillance devices and IoT devices to monitor the patient's activities, filtering out unimportant deviations from the patient's normal pattern of behavior, and sending reports to caregiver 180, and reminders to patient 110, to maintain the same level of care with less interruption of the caregiver's time. This reduction in in-person time provides caregiver 180 with more private time. The software's actions in collecting, analyzing, filtering and notifying caregiver 180 results in fewer actions by caregiver 180.

Routine reports on the patient's toilet activities, their use of toothbrush and soap, etc., can be delivered to caregiver 180 to be read at a time convenient to caregiver 180, rather than interrupting the caregiver's sleep or requiring a lengthy journey to visit patient 110.

IoT and other devices can monitor the patient's activities in bathing without recording or otherwise divulging their physical details or violating their privacy, yet still detecting through time measurement, use of soap dispenser, measurement of water used, and similar data, that patient 110 has or has not adequately bathed. Inadequate or abnormal use of bathing facilities, such as shower or bathtub, can be detected by the analysis system 160. Abnormal activity can then trigger a notification to caregiver 180, or a query through a digital assistant to patient 110, or both.

Machine learning and artificial intelligence in analysis system 160 can determine, through examination of video data observing patient 110, that the patient's gait, speed in walking, balance, and similar movements can be abnormal. If analysis system 160 determines an abnormal movement by the patient 110, this can trigger one or more notifications to caregiver 180 or nearby staff, or a query through digital assistant 190 to patient 110.

Caregiver 180 can become an active participant in a community of caregivers, all encountering and discussing common problems in delivering care to their patients. Caregiver 180 can be provided with reports from devices to maximize the benefits of such discussions. Analysis system 160 can direct the caregiver's attention to specific online resources, based on deductions from measurements of the patient's activities, thereby reducing the caregiver's actions in ineffective searching. Community interaction reduces the stress and isolation felt by caregiver 180.

The patient's successful use of diapers, toilet 264, or general cleanliness—all of which can be monitored by IoT devices—can be reported after the fact to caregiver 180. Only the situations in which patient 110 is not performing adequate self-care can require the attention and time of caregiver 180.

Other databases 17 may be accessed to improve patient care or to optimize the caregiver's time with patient 110. Data collected from patient 110 can be correlated to other population data to detect anomalies. For examples, a purchasing history of the patient to detect changes in supplies purchased and used (e.g., new brands, new styles), travel history (e.g., to and from appointments, social events), etc. In an example, patient 110 may go shopping and procure supplies, this event triggering a notification to caregiver 180 that their assistance may be needed in putting the procured supplies into the proper locations, such as medical supplies into an IoT-equipped medicine cabinet. Further, in reviewing the types of supplies procured, such as proper over-the-counter medicines or model of adult diapers, and in updating the database on the quantities of supplies available to the care receiver.

Referring to FIGS. 1 and 2, patient 110 of FIG. 1 can also be user 220 of the monitoring system in FIG. 2.

In some examples, patient 110 is monitored by one or more monitoring devices 120, which can be supplemented by one or more fitness devices 122, and one or more smart patches 124 worn by the care receiver.

In examples, a “smart” patch 124 is attached to the medial malleolus of patient 110 on the right or left ankle of patient 110 in accordance to examples. The placement of each patch 124 is designed to cause electrical stimuli to activate the tibial nerve of patient 110 in one example to alleviate overactive bladder (“OAB”) symptoms. The term “smart”, in general, refers to the use of memory and logic components and instructions, and may also include electronic components for communications, to generate some or all of the functionality disclosed herein.

Patch 124 can be any type of device that can be fixedly attached to patient 110 and includes a processor/controller and instructions that are executed by the processor, or a hardware implementation without software instructions, and communication elements to provide communications with controller 130 in some examples. Patch 124 can also include additional components that provide topical nerve stimulation on patient 110 to provide benefits to patient 110, including bladder management for an overactive bladder, such as electrodes, sensors, a battery, adhesive, a control unit, an electronic integrated package, stimulators, etc.

Patch 124 in one example can include a flexible substrate, a malleable dermis conforming bottom surface of the substrate including adhesive and adapted to contact the dermis, a flexible top outer surface of the substrate approximately parallel to the bottom surface, one or more electrodes positioned on the patch proximal to the bottom surface and located beneath the top outer surface and directly contacting the flexible substrate, electronic circuitry embedded in the patch and located beneath the top outer surface and integrated as a system on a chip that is directly contacting the flexible substrate, the electronic circuitry integrated as the system on the chip and including an electrical signal generator integral to the malleable dermis conforming bottom surface configured to electrically activate the one or more electrodes, a signal activator coupled to the electrical signal generator, a nerve stimulation sensor that provides feedback in response to a stimulation of one or more nerves, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to activate/stimulate one or more nerves of a user wearing patch 124 at least at one location proximate to patch 100. Additional details of examples of patch 124 are disclosed in U.S. Pat. No. 10,016,600, entitled “Topical Neurological Stimulation”, the disclosure of which is hereby incorporated by reference.

In some examples, the monitoring devices, such as patch 124, can send and receive data from controller 130, and send the data on to cloud service 140. In some examples, caregiver 180 can access social media site 150, and communicate with one or more social media participants 152.

In some examples, patch 124 stimulates the tibial nerve of patient 110 at the direction of caregiver 180 via direct or remote activation to elicit a suppressive nerve response, which, in turn, suppresses the urination impulse.

In some examples, the system measures the state of the patient's bladder to determine the degree of urgency in voiding the bladder. In some examples, the system uses ultrasound to measure the state of the bladder.

In some examples, the system may measure other biometric attributes of patient 110 to determine the degree of urgency in voiding the bladder. Examples of these measurements may be a clenching of abdominal muscles, or restlessness during sleep, or the shape or opacity of the bladder when imaged.

Biometrics refers to body measurements and calculations and metrics related to human characteristics. Biometric identifiers are the distinctive, measurable characteristics used to label and describe individuals and include physiological and behavioral characteristics. Physiological characteristics are related to the shape of the body. Examples include veins, face recognition, DNA, palm print, hand geometry, iris recognition, retina and odor/scent. Behavioral characteristics are related to the pattern of behavior of a person, including typing rhythm, gait, and voice.

In some examples, the monitoring data can be analyzed by analysis system 160, and reported to caregiver 180 or to the patient 110, or to others, by reporting system 170. In some examples, analysis system 160 writes and reads information from server 162, which may be connected through the cloud service 140 or directly to the analysis system 160.

In some examples, analysis system 160 accesses health data and population data from health database 164, which provides data for machine learning and analysis. In some examples, monitoring device 120 can capture visual data, or audio data, or presence data rendered as metadata, or combinations of these types of data.

In some examples, digital assistant 190 can recognize voice commands or gestures, or both, from patient 110, and can output audio or visual feedback, or both, from analysis system 160 or from caregiver 180 to patient 110. Further, digital assistant 190 can use visual means, with touch screen or buttons, to interact with patient 110. Video interaction between patient 110 and caregiver 180, such as a scheduled video call with a family member, can collect data or be used to monitor the patient's condition. Interfacing with digital assistant via “audio means” includes the use of tones or buzzing, such as in a necklace or amulet type of device, this device being able to notify patient 110 when necessary, such as time to take medicine, or notification of an incoming phone call on a dedicated device remote from the patient's current location.

In some examples, patient 220 visits bathroom 210 and is monitored by one or more of wireless fitness device 230, weight monitoring device 232, monitoring mirror 234, monitoring camera 236, and audio input device 240. In some examples, patient 220 can have their actions measured by sink 260 with faucet 262, for water consumption and timing; by toilet 264, by recording time on toilet 264; by monitoring tooth brushing duration using the smart toothbrush; and by toilet tissue dispenser 268, for recording an amount of paper used. In some examples, patient 220 can interact with analysis system 160, or caregiver 180, or both, through audio or visual means using digital assistant 190. In some examples, accessories can include wireless accessories 270 or non-wireless accessories 280.

Although a single patient 110 is generally disclosed above, examples can optimize the caregiver's time when caring for multiple patients 110. In an example, caregiver 180 may be responsible for multiple patients care in a caregiving facility. The system can prioritize the notifications by condition and by patient, and even by location of the patient, to optimize the use of the caregiver's time while in the facility. This triage step may be performed by the system using artificial intelligence or machine learning. Similarly, the caregiver's travel to multiple locations can be optimized, and the standard of care given to patients optimized, by the system plotting the route and list of activities for caregiver 180.

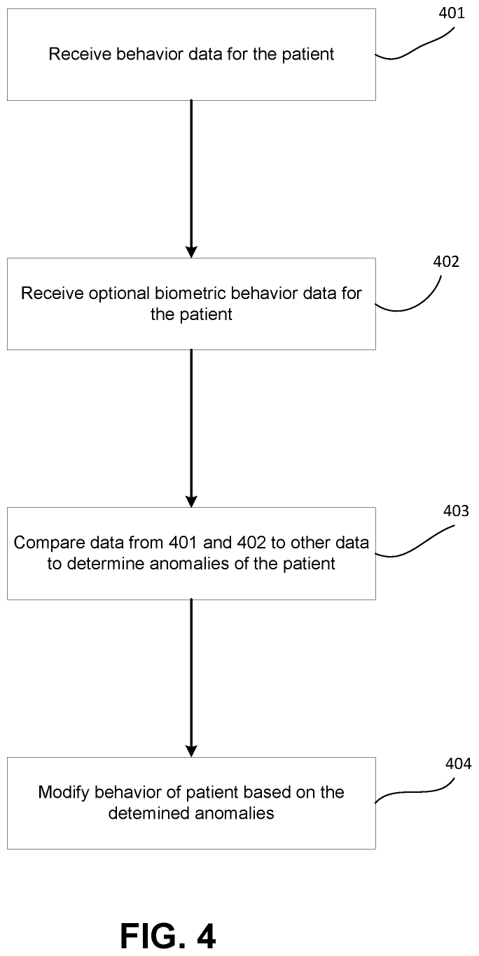

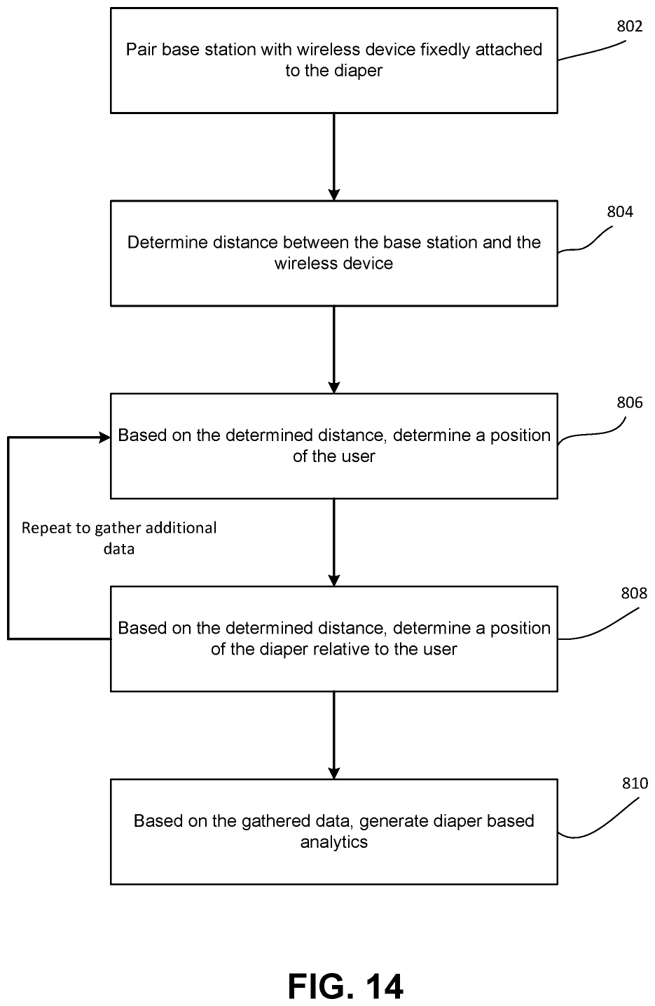

FIG. 4 is a flow diagram of the functionality of one or more of the elements FIGS. 1-3 for behavior monitoring and modification of a patient as disclosed herein. In one example, the functionality of the flow diagram of FIG. 4 (and FIG. 14 below) is implemented by software stored in memory or other computer readable or tangible medium, and executed by a processor. In other examples, the functionality may be performed by hardware, for example through the use of an application specific integrated circuit (“ASIC”), a programmable gate array (“PGA”), a field programmable gate array (“FPGA”), etc., or any combination of hardware and software.

At 401, behavior data for the patient is received. The behavior can include, for example, use of bathroom related elements such as shown in FIG. 2, including a toothbrush, medical cabinets or medicine dispensers, toilet use, diaper use, etc., movements of the patient, shopping patterns of the patient, etc. The bathroom related behavior that is monitored in examples using the elements of FIG. 2 include:
toilet use, including time sitting, toilet paper use, washing at the sink by measuring water use at the tap and soap use at a dispenser;
bathing frequency and duration by measuring water use, soap use, timing of shower door opening and closing;
brushing of teeth by measuring toothbrush use, toothpaste use by frequency of repurchase or restocking, camera monitoring;
hair care by measuring shampoo use, hair brush use;
clothing use by measuring clothes as they are removed from dresser, and how they are put into laundry (e.g., using RFID tags);
incontinence by measuring soiled clothes (e.g., water detection), camera tracking.

At 402, optional biometric data for the patient is received. The biometric data can include:
temperature, bladder condition, etc. heart rate, blood pressure, blood oximetry, glucose;
arrhythmia incidents, such as atrial fibrillation;
spoken words, to detect slur from stroke;
EEG, EMG, ECG;
long-term posture changes, walking gate, walking posture, foot dragging; in some examples, posture is determined using the smart diaper system disclosed below;
counts of various activities, such as standing up from sitting, step counts, toilet usage.

At 403, the data collected at 401 and/or 402 is compared to historical data of the patient and/or of a general population, to detect abnormal activities or anomalies of the patient. Examples correlate to demographic data across age, gender, BMI, medical conditions such as diabetes or sleep apnea. Examples further correlate to the patient's own historical data, such as weight, calorie intake, steps taken per day, sleep hours and quality of sleep, heart data, blood data and blood pressure data.

At 404, based on the detection at 404, the caregiver can take actions to modify the behavior of the patient or otherwise response. The monitoring of data may inform the patient, the caregiver, the care provider and local staff such as medical professionals. This information may lead to positive reinforcement as well as corrective action. Examples of modifying include:
Monitor toilet use and sink use, correlate to eating schedule and data, exercise schedule and data, modify the person's use of the toilet, use of toilet paper, use of washing. Notify caregiver of frequency and adequacy of hygiene;
Monitor dental care, correlate to data from dentist, cohort data (similar population). Modify behavior by new diet rules;
Monitor hair care, and notify caregiver. Modify behavior by coaching, new hair style with ease of use, new diet;
Monitor changes of clothing and notify caregiver;
Monitor incidents of incontinence and notify care provider as well as caregiver. Modify behavior by fewer outings, more visits to the toilet, coaching in self-cleaning.

Smart Diaper System

In conjunction with the above, in some examples a “smart” diaper is used to monitor movement and the duration of use of the diaper on the patient's body and collect data over time. The collected data can provide analytics, including determining a posture of the user and other behavior of the patient, that is used as additional inputs to monitoring system 100 disclosed above.

Through the collection of data as to the position of the diaper on the user's body across a span of time, and the collection of data as to the user's bladder or bowel movements across a similar coincident span of time, the occurrence of bladder or bowel activity may be correlated to the various wearing positions of the diaper and the disposal of the diaper, such that the number of diapers being used across a span of time may be reduced, and the choice of a particular style of diaper such as overnight or out of the home diaper may be optimized for the user.

Further, collection of data as to the position of the diaper on the user's body across a span of time, such as 24 hours, may show by analysis of the data a pattern of repeated trips to the bathroom even though the diaper is not replaced. Such repeated trips indicate small or non-existent urine or bowel discharges, and may indicate recurring urges to empty the bladder or bowel when emptying is not necessary. Reducing trips to the bathroom can provide substantial benefits to the user, such as by reducing interruptions to work during the workday, and reducing the danger of falls and interruptions to sleep during the night. Further, this data may be used in combination with an electrical stimulator that stimulates nerves to reduce the urge to urinate or have a bowel movement, thus reducing and/or delaying the repeated trips.

The term “smart”, in general, refers to the use of memory and logic components and instructions, and may also include electronic components for communications, to generate some or all of the functionality disclosed herein.

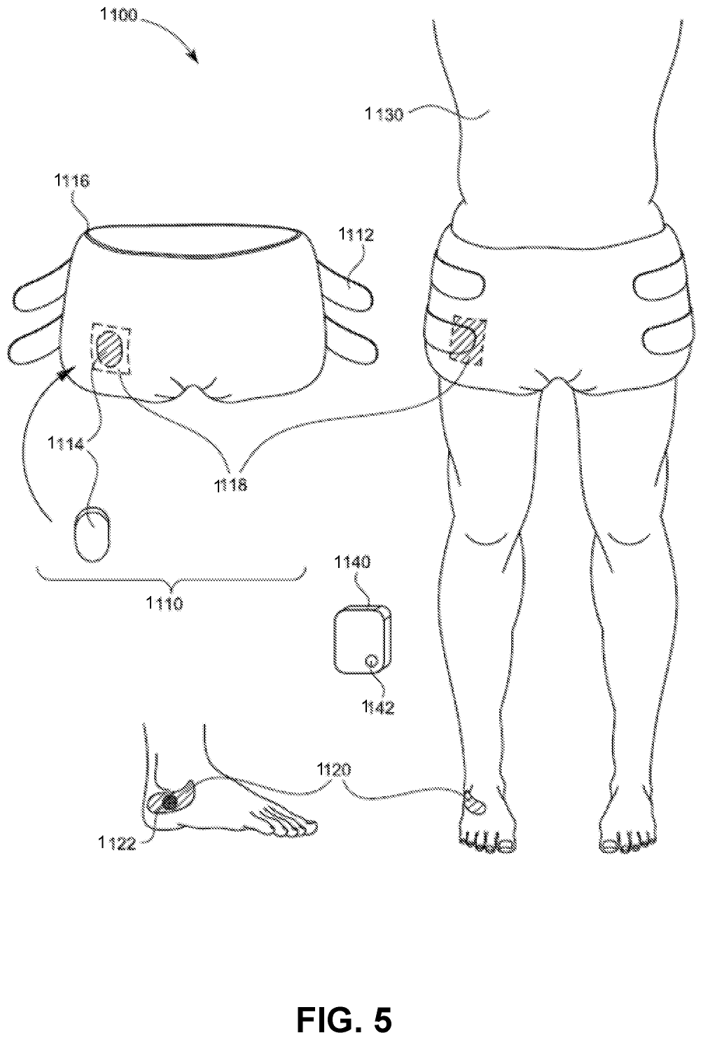

FIG. 5 illustrates an example smart diaper and data collection system 1100. System 1100 includes a diaper 1110 with a diaper body that includes an absorbent pad, tabs 1112, a wireless beacon or monitoring device (generally, a “wireless communication device”) 1114 fixedly attached to the diaper body, a waistband 1116 and a beacon holder 1118. System 1100 further includes a smart band aid or smartpad or patch 1120 (hereinafter, “patch”), worn at the ankle or other fixed position, and an optional patch annunciator 1122. An optional host device 1140 and an optional host device annunciator 1142 are further associated with a user 1130. In the example of FIG. 1 and other examples disclosed below, user 1130 is an adult and diaper 1110 is an adult diaper.

Wireless beacon 1114 can be any type of wireless communications device or monitoring device that uses wireless technology and protocols including Radio-frequency identification (“RFID”), Near-field communication (“NFC”), Wi-Fi, Bluetooth Low Energy (“BLE”), or others. Wireless beacon 1114 is fixedly attached to diaper 1110.

Patch 1120 can be any type of device that can be fixedly attached to a user and includes a processor/controller and instructions that are executed by the processor, or a hardware implementation without software instructions, and communication elements to provide communications with wireless beacon 1114. Patch 1120 can also include additional components that provide topical nerve stimulation on the user to provide benefits to the user, including bladder management for an overactive bladder. In examples, patch 1120 is the same type of patch as patch 124 of FIG. 1.

Host device 1140 can be a smartphone or a fob, or any device with communications capability to provide communications with wireless beacon 1114 and/or patch 1120. Host device 1140 may also include applications (“apps”) or other types of user interfaces to allow a user to control wireless beacon 1114 and/or patch 1120.

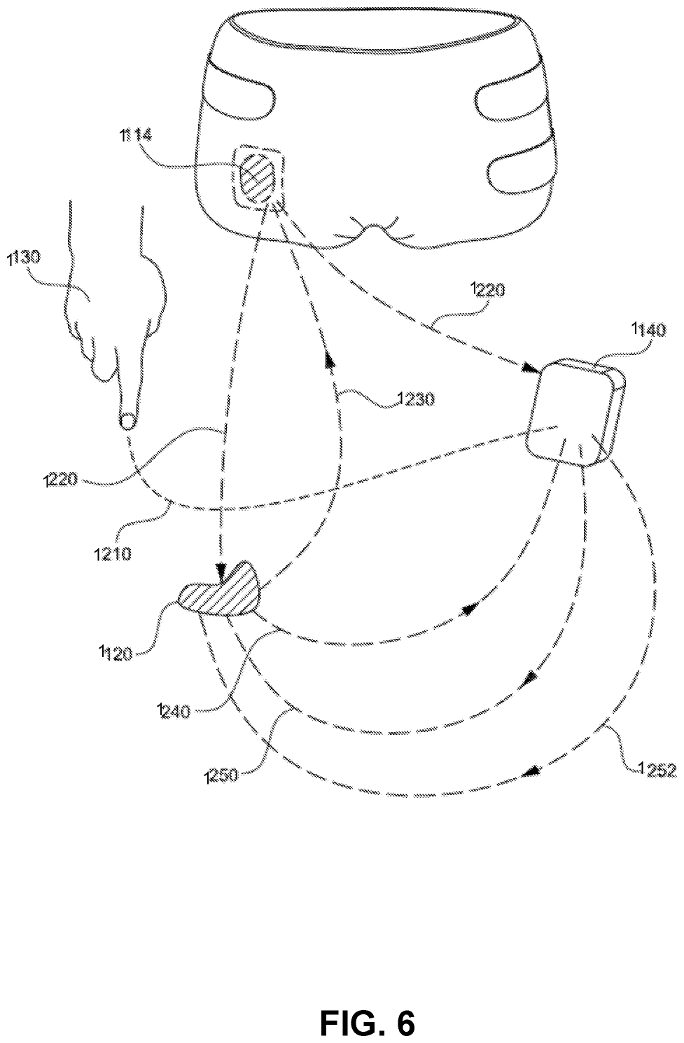

FIG. 6 illustrates various examples of communication signals of data collection system 1100 of FIG. 5. The signals include a user signal 1210 from user 1130 to host device 1140, a beacon data signal 1220 from wireless beacon 1114 to host device 1140, a control signal 1230 from patch 1120 to wireless beacon 1114, and a data signal 1240, a host control signal 1250 and a host data signal 1252 between patch 1120 to host device 1140.

Patch 1120 and beacon 1114 establish communication using wireless means, such as Bluetooth based communications in all its variations, Wi-Fi, or other RF means.

In some examples, host device 1140 establishes communication using wireless means with one or both of patch 1120 and beacon 1114.

In some examples, patch 1120 controls beacon 1114 using control signal 1230, and beacon 1114 sends data to patch 1120 using beacon data signal 1220.

In some examples, host device 1140 controls beacon 1114 using host control signal 1250, and beacon 1114 sends data to host device 1140 using beacon data signal 1220.

In some examples, host device 1140 and beacon 1114 are part of a larger Body Area Network (“BAN”) that includes additional smart devices, such as fitness trackers, smart watches, smart phones, smart tags, and similar means.

Diaper 1110 is worn by user 1130 at various positions while the user is standing, including at the waist, at the thighs, at the knees, at the calves, at the feet, and separate from the body. User 1130 at various positions wears diaper 1110 while the user is sitting, including at the waist, at the thighs, at the knees, at the calves, and at the feet.

In some examples, data collection is performed by measuring a distance between beacon 1114 and the patch 1120. In some examples, data collection is performed by measuring a distance between the beacon 1114 and the host device 1140. In some examples, data collection is performed by measuring distances among beacon 1114, the patch 1120, host device 1140, and other beacon equipped devices.

Data collection system 1100 distinguishes the standing positions from the sitting positions in order to distinguish through measurement and analysis of the data the occurrence of a sitting event from a non-sitting event.

Data collection system 1100 may be trained by user 1130 to recognize specific positions/postures of the user, such as standing, sitting, lying down, with the pad in various specific positions, such as at the waist, on the thighs, at the knees, at the calves, by the user confirming via signal from the user to data collection system 1100 when data collection system 1100 indicates a position matching the actual position of the user. Based on the saved data, system 1100 can determine a future position/posture of the user using current data of distance between beacon 1114 and patch 1120, or any other fixed host device.

Data collection system 1100, and analytical protocols applied to the collected data, may be used to analyze patterns of behavior of the user of the diaper 1110 to obtain analytics. This analysis may be used to calculate usage patterns for diaper 1110, and to predict the next usage event, including but not limited to wearing a new diaper, voiding fluids or solids into a worn diaper, using a toilet after pulling the diaper down, disposing of a diaper and removing a diaper when not near a toilet.

In some examples, analysis of the data may reveal a pattern of discarding or disposing of a used diaper without events related to voiding wastes, indicating use of a diaper without soiling the pad.

In some examples diaper 1110 is designed to be pulled up and down the legs as a pair of undershorts using waistband 1116, so that the diaper does not include tabs 1112.

In some examples, diaper 1110 is designed to be wrapped around the user's body, using tabs 1112 to attach the front of the diaper to the back of the diaper so that the diaper remains at the user's waist while in use. In these examples, diaper 1110 may require that the tabs 1112 be loosened in order that the user may move the diaper down or up the legs.

In some examples, data collection and analysis leads to improvements in selection of different types of diapers 1110 for different use cases, such as a thinner diaper/pad for comfort and less-obtrusive wear during the day, compared to a thicker diaper/pad for overnight use and use away from home.

In some examples, data collection and analysis tracks diaper 1110 usage to inform the user when supply of unused diaper is low, the count of unused diapers being reset to a fixed number by the user, such as when additional diapers are purchased, and the count being reduced by one each time data collection system 1100 determines, through analysis of data, that a diaper 1110 has been disposed of.

In some examples, diaper 1110 is designed to allow the user to insert and remove beacon 1114 from beacon pocket 1118, beacon pocket 1118 being designed to accommodate the size of beacon 1114 while enclosing beacon 1114 securely so that beacon 1114 does not come loose from beacon pocket 1118, nor does beacon 1114 become inadvertently separated from the diaper 1110.

In some examples, beacon pocket 1118 holds a pre-installed beacon 1112, the pocket not being designed to allow the user access to the enclosed beacon 1112. In other embodiments, beacon 1112 is integrated, disposable, and permanently attached to diaper 1110.

In some examples, patch 1120 and/or host device 1140 collect other data, in addition to distance measurement data. Other data may include time stamps, date stamps, device power level indications, body temperature, pulse rate, circadian rhythm indicators, and other biometric data. Analysis of such data may provide correlation of movements of the diaper 1110 with levels of activity, such as contrasting sleeping, eating at rest, walking, sports, and the like.

Other data may be geographical coordinates indicating the user's location while wearing diaper 1110. Analysis of such data may provide correlation of movements of the diaper 1110 on the body with geographical locations, such as the user's home, the user's place of work, travel long distances away from home, and the like.

Analytical protocols in patch 1120 and/or host device 1140 determine the position of beacon 1114 and the position of diaper 1110 relative to the position of patch 1120 or the position of host device 1140 or both, to process position data. Analytical protocols may process a series of position data to determine patterns in the measured values, thereby deriving dependable indications of diaper 1110 positions.

In some examples, if patch 1120 detects intermediate distances to beacon 1114, such as the sitting thigh position, or the sitting knee position, patch 1120 may prompt the user, such as through annunciator 1122 or host device annunciator 1142, to push diaper 1110 further down or pull it further up so that the distance and inferred position is not ambiguous as to correlation with using the toilet.

When the user carries host device 1140 with them in a repeatable location, such as a shirt pocket, then the distance from host device 1140 to beacon 1114 may be predictable according to the relative location of diaper 1110 to host device 1140.

When host device 1140 is installed in a fixed location, such as a power outlet near the user's toilet, host device 1140 can communicate in coordination with patch 1120 on the user's body, the data collection system annotating the distance measurements with the location information from the fixed host device. The data analysis protocols may use these annotations to filter out user position information from the bathroom from user position information from other rooms, such as sitting with diaper 1110 pulled down in the bedroom while changing clothing.

In some examples, host device 1140 is an element of an appliance such as an air freshener, which the user plugs into a power outlet in the bathroom. In some examples, the beacon 1114 has an integral battery. In some examples, the beacon 1114 has no integral battery, deriving the power to reply to the measurement signal from the measurement signal itself.

In some examples, BLE protocol is used to communicate between the patch 1120 and beacon 1114, or between host device 1140 and beacon 1114, or both, since BLE is able to estimate distance between two BLE-equipped objects to a sufficient accuracy adequate for this application.

In some examples, more than one beacon 1114 may be placed in diaper 110 at various distinct locations to increase the reliability of the measurement path through redundancy.

In some examples, more than one patch 1120 may be placed within range of beacon 1114 to increase the accuracy and reliability of the measurement path, such as wearing one medial patch 1120 on the forearm and wearing a tibial patch 120 at the ankle, each having connection to the others to coordinate distance data and to make a determination of the diaper 1110 position.

In some examples, system 1100 may include a set of diapers, one diaper 110 having a pre-installed beacon 1114, but such beacon 1114 being removable from diaper 1110 after the diaper is used and before the diaper is discarded, such that the beacon 1114 may be installed by the user in the next diaper in the set of diapers, this re-installation process including one or more steps such as pressing of a button on beacon 1114 to reset it each time it is installed or attached to a new diaper. This example system optimizes the use of one beacon 1114 across a set of diapers 1110 without reducing the efficacy of beacon 1114 as it is reused. Reuse of beacon 1114 reduces the number of disposed beacons 1114, and reduces the impact to waste management and recycling.

In some examples, system 1100 may include diapers with no included beacon 1114, the beacon 1114 being acquired separately by the user, such as through concurrent purchase with the diapers. The purchased diapers are diapers which are designed with a beacon holder 1118 to accommodate a variety of beacons 1114 of various sizes, such that the separately acquired beacon may be inserted by the user before using the diapers. In some examples, a set of beacons 1114 may be acquired separately from the set of diaper; each beacon 1114 designed to be used in one and only one diaper and then discarded. In some examples, one beacon 1114 may be acquired separately from the set of diapers, the beacon 1114 to be reused by the user in a series of diapers up to a maximum number of reuses.

In some examples, system 1100 may include diapers which are not specifically designed for use with a beacon 1114 with beacon 114 being acquired separately and fixedly attached to one or more diapers in succession through the use of a clip or adhesive or the like.

In some examples, beacon 1114 is activated when diaper 1110 is removed from its outer packaging, this activation being triggered by the removal of the outer packaging. In some examples, beacon 1114 is deactivated when its power source is depleted, such as a battery with insufficient power remaining to power the beacon.

Beacon 1114 may also be modified and applied to other locations on the diaper for purposes of being detected at a specific distance by patch 1120 and/or the host device 1140.

In some examples, the present invention may be used in trial situations with a limited user set, for the purpose of improving use of monitoring devices and/or adult diapers or both. In some examples, the present invention may be mass-produced to be distributed by caretakers to users, to improve the use of adult diapers for users.

In some examples, the present invention may be mass-produced to be purchased and used in a public marketplace, to improve the use of adult diapers for users.

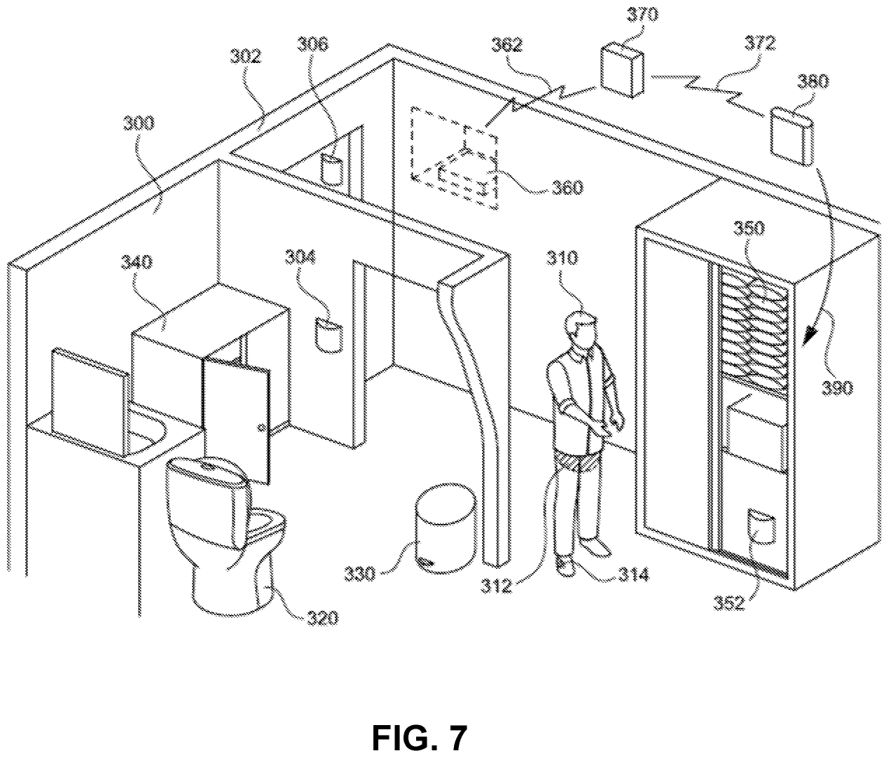

FIG. 7 illustrates an example environment for use with system 100. As shown in FIG. 7, data from a user 310 wearing a diaper-with-beacon 312 and a patch 314 is used on coordination with data collected when user 310 is in a bathroom 300, a bedroom 302 or other rooms in the user's living space. When user 310 is in bathroom 300 or bedroom 302 or other spaces, the position of user 310 and diaper-with-beacon 312 relative to patch 314 is monitored as described, and this information is related to a bathroom monitoring appliance 304 and a bedroom monitoring appliance 306 through a wireless protocol such as BLE, sending the data to a collection hub 360. Additional monitoring appliances, such as a local supply cabinet 340, monitoring appliance 304 and the supply closet 350 monitoring appliance 352 tracks the user's movements.

Disposal of the diaper-with-beacon 312, such as into disposal can 330 is recorded by patch 314, the data then being sent to collection hub 360. As a new diaper-with-beacon 312 is taken from supply cabinet 340 or supply closet 350, or other inventory location, and worn on the user's body, the action of wearing the diaper-with-beacon 312 is recorded by patch 314, the data for this event being sent to collection hub 360. Collected data is transmitted via wired or wireless means 362 to a local server 370 such as in the office space of an institution.

Extended collected data is transmitted via wired or wireless means 372 to a remote server 380. The inventory fulfillment system associated with or connected to local server 370 or remote server 380 then triggers the delivery of additional inventory/quantity 390 of diapers-with-beacons 312 to resupply storage closet 350. Analysis of derivations optimizes the reordering of pad supplies.

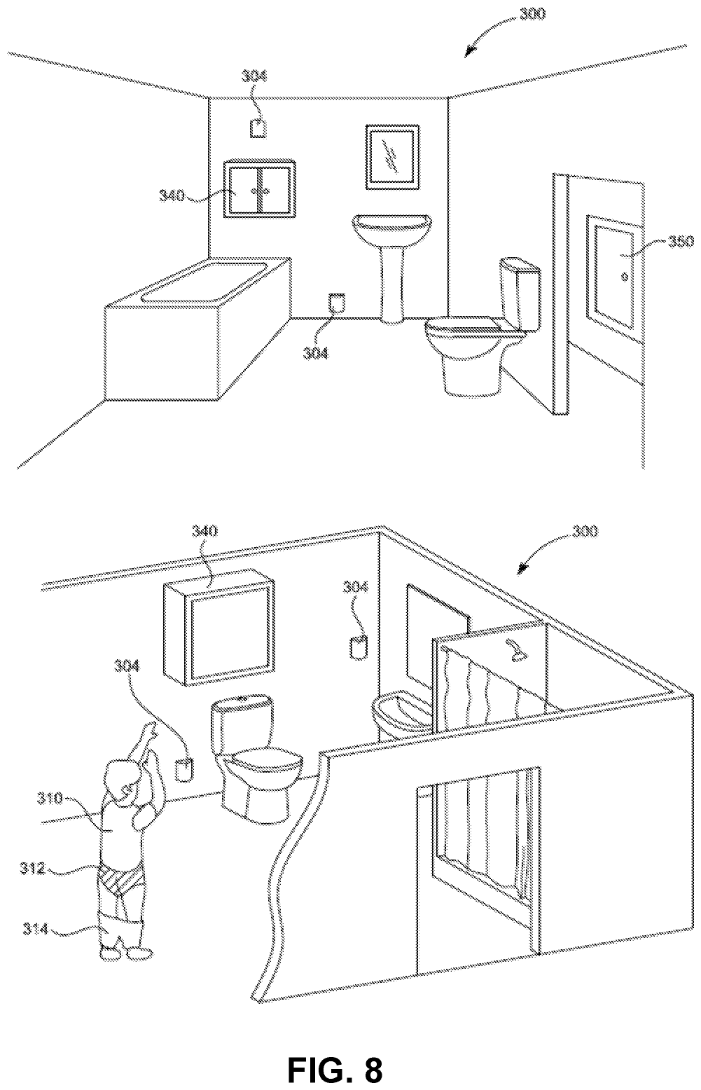

FIG. 8 further illustrates an example environment for use with system 1100. As shown in FIG. 8, the position of diaper-with-beacon 312 on user 310 may be determined with additional accuracy by using multiple bathroom monitoring appliances 304 arranged within the bathroom 300 or other rooms in the user's living space, such that the combination of measured distances among these monitoring appliances 304 and diaper-with-beacon 312 and patch 314 provides data establishing unique absolute distances, or ratios of distance, or a combination of absolute and relative measurements which are then processed by analytical means. The locations of these multiple bathroom monitoring appliances 304 may be selected to take into account the dimensions and arrangement of the bathroom and its appliances, such as sink, toilet, shower, tub, and others. Monitoring devices may also include Bluetooth toothbrushes, Bluetooth air fresheners (plugged into the wall), or dedicated Bluetooth beacons.

In some examples, the user is able to choose the combination of different types of diapers 1110 to be ordered or re-ordered when the diaper inventory 390 quantities fall below a minimum limit, thus providing the user an individualized service which establishes a relationship between the user and the supplier or suppliers, as well as restocking the inventory efficiently. This relationship, particularly with a single supplier who can fulfill the order for all requested types of diapers, is beneficial to the user in providing the user with familiar products, discounted offers, special delivery choices, and similar benefits, and beneficial to the supplier in providing access to a satisfied user for ongoing orders and as a customer for future products.

In some examples, wireless beacon 1114 may be inserted into a variety of diaper types used in the course of a day or week or similar period by a user, the identity of each wireless beacon 1114 being unique such that data collection system 1100 may count the number of diapers of each type used by the user. Patterns of usage may be discerned by data analysis, such as particular diaper types for particular times of the day, particular user behaviors including exercising, sleeping, traveling and particular dietary changes. Resupply of diaper inventory 390 may be improved by analysis of particular diaper type usage, including anticipating diaper usage according to future events, such as dietary changes, health changes, travel plans, etc.

In some examples, resupply of inventory 390 may be effective when a centralized inventory is tracked for the usage of diapers-with-beacons by a group of users, such as in an institution such as a senior living center. Correlation of diaper type usage per individual may be integrated numerically across the group of users to anticipate inventory demands of all diaper types in use.

In some examples, the ongoing measurement of diaper 1110 usage, transmitted to and collected by remote server 380, and analysis of the data, may provide one or more of the manufacturer and the distributor and the retailer of the diapers 1110 to improve the selection of diaper types in their manufacturing line or inventory or on store shelves, according to the aggregate usage data.

The system of diapers-with-beacon 312 working in combination with the collection of data to servers and analysis of that data by diaper 1110 providers may allow a change in behavior of the user by changing from refilling their diaper inventory by purchases at a local shop or online to an automated refilling process through the provider. In some examples, the user may opt-in to such an automated refilling system.

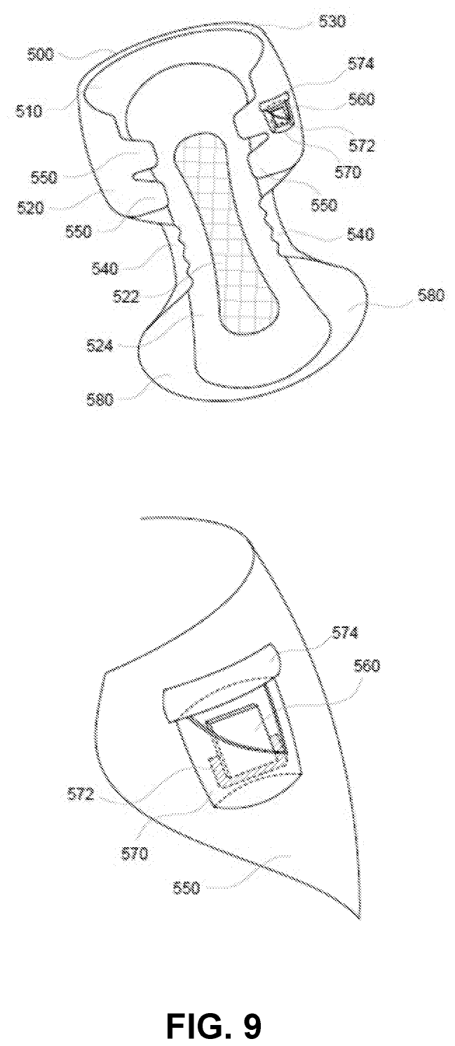

FIG. 9 illustrates another example smart diaper 500. Diaper 500 includes a body 510, an external layer 520, a primary acquisition layer 522, an absorbent layer 524, a waistband 530, leg gathers 540, tabs 550, an electronic monitoring device or wireless beacon 560, a device pocket 570 with a device pocket catch 572, a device pocket flap 574 and front wings 580. Device pocket 570 is designed to hold monitoring device 560 securely, and device pocket 570 is positioned on diaper 500 to be comfortable for the user in all positions of the user's body in normal wear, while also providing convenient access to device pocket 570 through device pocket flap 574 when diaper 500 is designed with a user-removable monitoring device 560. Monitoring device 560 and device pocket 570 are positioned on diaper body 510 so as to be protected from wetting of other parts of diaper body 510, whether through external layer 520 or through absorbent layer 524.

In some examples, diaper 500 encloses the waist of the user and is pulled up and down the legs as a pair of undershorts, the diaper therefore having no tabs 550.

In some examples, diaper 500 is designed to allow the user to insert and remove monitoring device 560 from device pocket 570 by opening device pocket flap 574 and securing monitoring device 560 to device pocket catch 572 when inserting the monitoring device, and detaching monitoring device 560 from device pocket catch 572 when removing the monitoring device.

In some examples, device pocket 570 is designed to not be opened by the user, and therefore has no device pocket catch 572, and no device pocket flap 574, the device pocket 570 fully enclosing monitoring device 560.

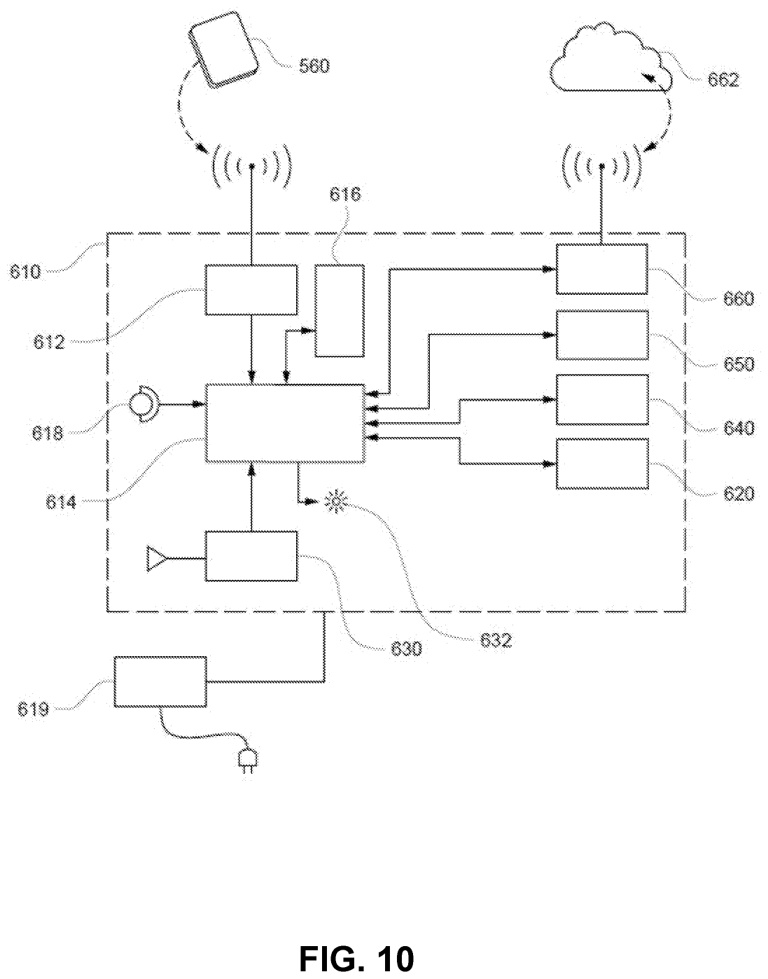

FIG. 10 is a block diagram of an example base station device 610 in communication with monitoring device 560. Monitoring device 560 communicates to base station device 610 using a base station data radio 612. Base station 610 includes a base station processor 614, a base station data memory 616, a base station enabling button 618, a base station power source 619, a data collection memory 620, a base station position indicator 630, an annunciator 632, an analysis processing device 640, a data archiving memory 650, and a cloud communication device 660 to provide communications to cloud memory 662. In some examples, annunciator 632 emits one or more of a visual signal, an audible signal or a vibration signal to the user.

Monitoring device position data, and other data, flows from monitoring device 560 via base station data radio 612 to base station device 610. Base station position data, and other data, flows from base station position indicator 630 to base station device 610. In base station device 610, the data from monitoring device 560 and from base station position indicator 630 is processed by analysis-processing device 640, under control of base station processor 614, and then is stored in memory. This memory may be any combination of base station data memory 616 and data collection memory 620 and data archiving memory 650 and cloud memory 662, data movement being coordinated by base station processor 614 according to the required use of the data. Data being of immediate use is stored in base station data memory 616. Data being of periodic on-device use is stored in data collection memory 620. Data being of occasional on-device use is stored in data archiving memory 650. Data is stored in cloud memory 662 for access by other devices through a network or other means.

Other data may include time stamps, date stamps, device power level indications, body temperature, pulse rate, circadian rhythm indicators. Analysis of such data may provide a correlation of movements of diaper 500 with levels of activities, such as contrasting sleeping, eating at rest, walking, sports, and the like.

Base station 610 is powered by base station power source 619.

Position data is processed by analysis-processing device 640 to determine the position of monitoring device 560 and the position of diaper 500 relative to the position of base station device 610. Analysis of pad position and use data is used to determine patterns of use.

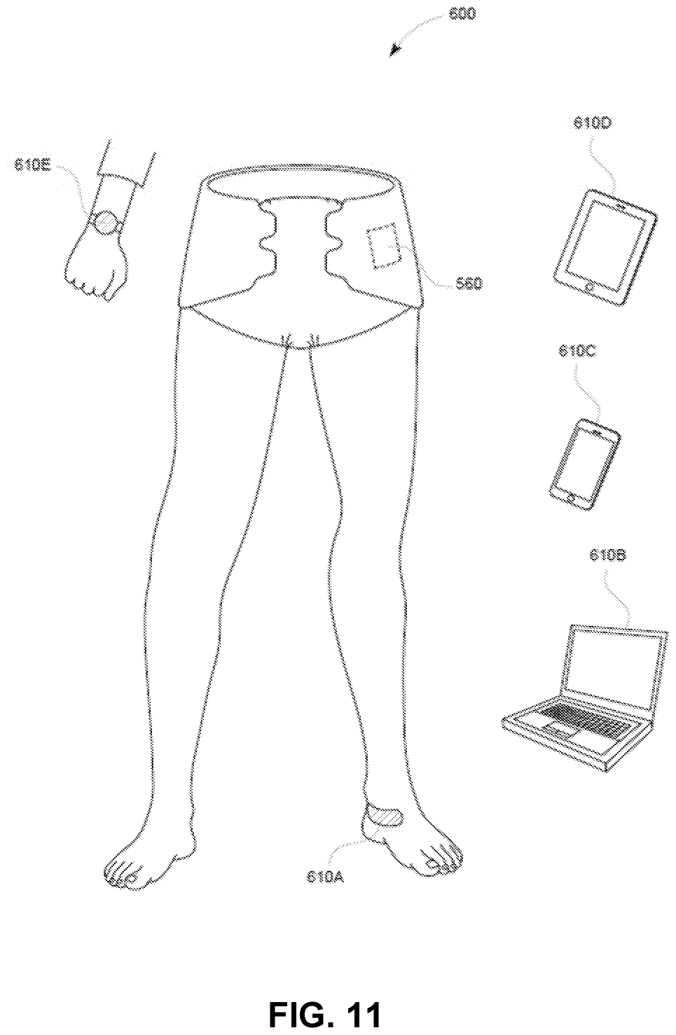

FIG. 11 illustrates an example smart diaper and data collection system 600. In FIG. 11, data collection system 600 includes base station 610 and monitoring device 560. The base station is one or more of a patch base station 610A, such as patch 1120 of FIG. 5, a custom base station 610B, a smartphone base station 610C, a smart pad base station 610D, and a smart watch base station 610E. Each of 610A through 610E includes some or all of the elements previously described in conjunction with base station 610 in FIG. 10. Further, in the example using patch base station 610A, patch 610A can include one or more of the elements described in conjunction with patch 1120, and vice versa.

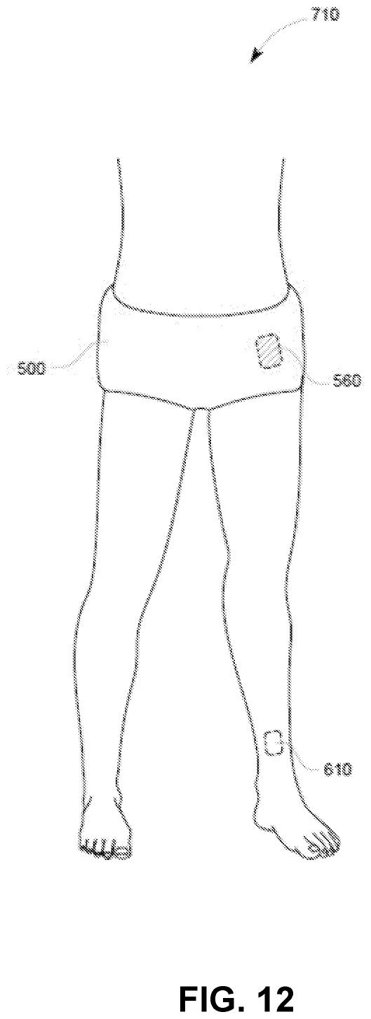

FIG. 12 illustrates a standing user with diaper 500 pulled up to the waist position 710, with monitoring device 560 positioned relative to base station 610.

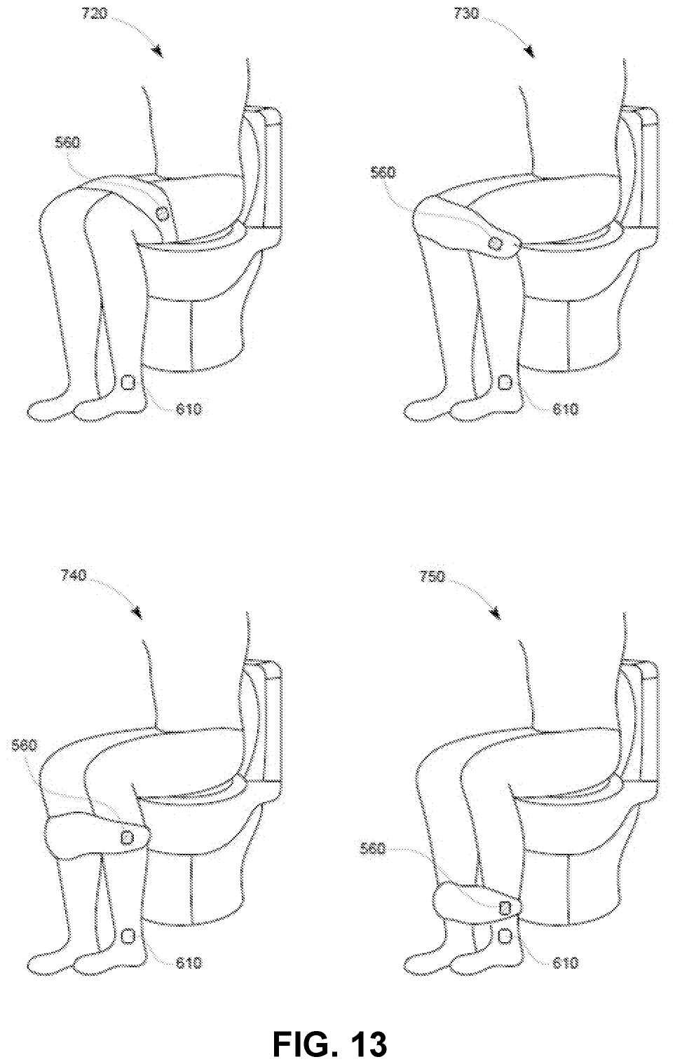

FIG. 13 illustrates four positions of monitoring device 560 relative to base station 610. Specifically, the seated user with diaper 500 pulled down to the thigh position 720; the seated user with diaper 500 pulled down to the knee position 730; the seated user with diaper 500 pulled down to the calf position 740; and the seated user with diaper 500 pulled down to the feet position 750. All of these positions can be detected through the communication interaction between monitoring device 560 and base station 610.

In some examples, if base station 610 detects intermediate distances to monitoring device 560, such as the thigh position 720 or the knee position 730, base station 610 may prompt the user, such as through annunciator 632, to push diaper 500 further down or pull it further up so that the distance and inferred position is not ambiguous as to correlation with using the toilet.

When the user carries base station device 610 with them in a repeatable location, such as a shirt pocket as for custom base station 6108 or patch base station 610C or smart phone base station 610D, or the user wears base station device 610 on their person, such as patch base station 610A or smart watch base station 610E, then the distance from base station device 610 to the monitoring device 560 will be predictable according to the relative location of diaper 500 to base station device 610.

In some examples, the analysis is simplified by using information input from the user of diaper 500, this information being input into base station device 610 to indicate to the base station to retrieve sensor position data from monitoring device 560 at various positions of the first use of diaper 500, such as the pulled-up and waist position 710, and the sitting and feet position 750. After the user confirms to base station processor 610 each of the distances corresponding to multiple positions, base station processor 610 can operate with measured distances from monitoring device 560.

Using disclosed examples, notifications of diaper use, bathroom use, such as when user enters bathroom, uses toilet, etc., may be sent to a third party such as a relative or a caregiver.

Each Bluetooth device that communicates with beacon 1114 or monitoring device 560 can respond to a “ping” and offer a “service.” Bluetooth device categories may be registered online, vendor-non-specific, etc.

Examples of behavior modification of the user, which may result from data monitoring, is to use fewer diapers. Further, where patch 1120 is a neural stimulator, the data may inform patch 1120 to perform stimulations at times of the day when a diaper change is recorded. For example, if diapers are being changed more frequently than is the norm, then the system assumes a leak is occurring. To forestall the leaking, a stimulation may defer urination. This means the use of fewer diapers. An app, for example on a smartphone or fob, may also inform the user directly to go and use the toilet before an anticipated urge occurs.

A discarded diaper may be added to the “used diaper list” in the software. The app (i.e., software) makes this determination when it sees a previously worn diaper's location move far away from the user. When that distance exceeds the “body length”, then the software assumes the diaper has been left behind/discarded. It is not necessary for the user to immediately unpack and put on a new diaper for that new diaper to replace the old diaper's “wearing this now” status. The user may not put on a new diaper immediately, but the app provides valuable data by recording “I took off a diaper” as described above. The user may also be instructed to touch a button or other means in the app, whenever they change a diaper.

Devices around the bathroom or other room may be added by the user. When a new device is detected by the app or fob or directly by the patch, it is useful to know the type of device. The app prompts the user to select the new device's “type” and “brand” in the app itself using, e.g., drop-down menus. Further, a caregiver or other person can make the same entries on behalf of the user, then hand the phone or fob back to the user.

As disclosed, examples determine a distance between beacon 1114 and patch 1120, as well as a distance in some examples between patch 1120 and host device 1140, which may be a fob, smartphone, or other base station devices as shown in FIG. 12. In a fob example, the distance is determined using BLE and the Received Signal Strength Indicator (“RSSI”). The Fob will attempt to pair with a patch 1120 it detects with the strongest RSSI signal, only if the patch is within 2 feet or some other predefined distance.

After the fob pairs with a patch, that connection is “latched”. The fob will pair with no other patch. The patch will pair with no other fob. Each device “remembers” the unique ID of the device with which it is paired.

If the BLE connection is broken, the fob will attempt to pair again, yet only complete a pairing with the patch it had immediately before.

Other patches that may reply to the “ping” of the fob searching for its partner will not be recognized by the fob software.

The fob pings the patch periodically as a kind of “heartbeat” to make sure that the patch is still there. Therefore, the fob knows within a margin of error that the patch is still there. This avoids the fob having to initiate a new pairing when the user presses the START button for a stimulation (i.e., when patch 1120 provides neural stimulation). The stimulation can begin immediately, without the “5 second” delay or other predefined amount of delay needed to complete a pairing sequence. Users do not want a delay of several seconds when they press START and expect the stimulation to start right away.

If the fob detects that it has a low battery level, it saves the ID information, state and strength of the connected patch. This data is saved into nonvolatile memory in the fob. The power for the fob can then be fixed. The Fob can then immediately re-pair with the patch it used before, assumed still to be in range and on the user.

If the fob fails to pair with a patch, then the fob tries again. This is useful when a patch moves out of range of its fob. This is repeated, but the time between attempts increases slowly, to the point where the fob gives up. This saves power by reducing the number of “pings.” If a ping is successful, then the fob checks the ID of that patch, and the patch checks the ID of the fob. If they are as remembered from the most recent paired condition, then the connection is reinstated.

As with pairing the fob to the patch, similar procedures can be used to pair beacon 1114 to patch 1120, and similar procedures can pair a smartphone or other host device to beacon 1114 and/or patch 1120.

FIG. 14 is a flow diagram of the functionality of a base station and/or host device (e.g., patch 1120, host device 1140, base station 610) when determining smart diaper based data and analytics.

At 802, the base station is paired with the wireless beacon 1114 or other wireless device fixedly attached to a diaper that is worn by a user.

At 804, a distance between the base station and wireless beacon 1114 is determined.

At 806, based on the determined distance, a position/posture of the user may be determined. For example, determined positions may include standing, sitting, lying down, etc. For example, previous positions based on previously determined distances and that have been confirmed by the user are stored. The determined distance is then generally matched to previously determined distance (e.g., the closest to the current determined distance) and the corresponding previous position is selected.

At 808, based on the determined distance, a position of the diaper relative to the user is determined. For example, determined positions may include at the waist, on the thighs, at the knees, etc.

806 and/or 808 are repeated during a timeframe to gather additional data.

At 810, based on the gathered data, diaper based analytics are generated. The analytics can be based on additional data such as the geographic locations of the user, including movement history, the occurrence of bladder or bowel activity, and prior usage history of the user. Generated analytics can include one or more of: usage patterns of the diaper, a prediction of a next usage event, selection of type of next diaper depending on use case, ordering of additional diapers, determining alerts to be generated, user behavior, the posture of the user, a determination to stimulate nerves of the user to prevent a need to urinate, etc.

Several examples are specifically illustrated and/or described herein. However, it will be appreciated that modifications and variations of the disclosed examples are covered by the above teachings and within the purview of the appended claims without departing from the spirit and intended scope of the invention.

What is claimed is:

1. A method of monitoring a patient, the method comprising:
generating current behavior data for the patient;
first comparing the current behavior data to historical behavior data for the patient;
based on the first comparing, determining one or more first anomalies of the current behavior data; and
in response to the determined first anomalies, modifying the behavior of the patient.

2. The method of claim 1, further comprising:
generating current biometric data for the patient;
second comparing the current biometric data to historical biometric data for the patient; and
based on the second comparing, determining one or more second anomalies of the current biometric data;
the modifying the behavior of the patient further in response to the determined second anomalies.

3. The method of claim 1, the behavior data generated by bathroom related devices and comprising one or more of: toilet use, sink use, bathing frequency, toothbrush use or hair care.

4. The method of claim 2, the biometric data comprising one or more of: temperature, bladder condition, heart rate, blood pressure, blood oximetry, glucose, spoken words or posture.

5. The method of claim 1, further comprising:
third comparing the current behavior data to historical behavior data for a population of people comparable to the patient.

6. The method of claim 1, further comprising notifying a caregiver of the first anomalies.

7. The method of claim 4, the biometric data comprising bladder condition, the method further comprising:
affixing a smart patch to an ankle of the patient;
the modifying the behavior comprising generating an electrical stimulation from the smart patch to modify the patient's urge to urinate.

8. The method of claim 4, the biometric data comprising posture that is determined from affixing a smart patch to an ankle of the patient and determining a distance between the smart patch and a wireless communication device fixedly attached to a diaper worn by the patient.

9. The method of claim 7, the smart patch comprising:
a flexible substrate comprising adhesive on a first side adapted to adhere to a dermis of the patient;
an electronic package directly coupled to the substrate, the electronic package comprising a control unit and one or more stimulators; and
electrodes directly coupled to the substrate and the electronic package and disposed between the adhesive and the dermis.

10. A patient behavioral monitoring system comprising:
a plurality of behavioral monitoring devices in communication with a network;
an analysis system in communication with the behavioral monitoring devices over the network that receives current behavior data for the patient, the analysis system configured for first comparing the current behavior data to historical behavior data for the patient and based on the first comparing, determining one or more first anomalies of the current behavior data; and
a modification device that, based on the determined anomalies, is configured to modify the behavior of the patient.

11. The system of claim 10, the behavioral monitoring devices comprising at least one of: a toilet use monitoring device, a sink use monitoring device, a bathing frequency monitoring device, a toothbrush use monitoring device or a hair care monitoring device.

12. The system of claim 10, further comprising:
a plurality of biometric data monitoring devices in communication with the network;
the analysis system in communication with the biometric data monitoring devices over the network that receives current biometric data for the patient and further configured for second comparing the current biometric data to historical biometric data for the patient and based on the second comparing, determining one or more second anomalies of the current biometric data; and
the modification device further configured to modify the behavior of the patient in response to the determined second anomalies.

13. The system of claim 12, the current biometric data comprising one or more of: temperature, bladder condition, heart rate, blood pressure, blood oximetry, glucose, spoken words or posture.

14. The system of claim 11, the modification device notifying a caregiver of the patient of the first anomalies.

15. The system of claim 13, the plurality of biometric data monitoring devices comprising a smart patch affixed to an ankle of the patient;
the modification device generating an electrical stimulation from the smart patch to modify the patient's urge to urinate.

16. The system of claim 13, the biometric data comprising posture that is determined from affixing a smart patch to an ankle of the patient and determining a distance between the smart patch and a wireless communication device fixedly attached to a diaper worn by the patient.

17. The system of claim 15, the smart patch comprising:
a flexible substrate comprising adhesive on a first side adapted to adhere to a dermis of the patient;
an electronic package directly coupled to the substrate, the electronic package comprising a control unit and one or more stimulators; and
electrodes directly coupled to the substrate and the electronic package and disposed between the adhesive and the dermis.

18. The system of claim 17, the smart patch comprising a processor and a communication device and functioning as a centralized communication hub for the behavioral monitoring devices and the biometric data monitoring devices.

19. A computer-readable medium storing instructions which, when executed by at least one of a plurality of processors, cause the processor to monitor a patient, the monitoring comprising:
generating current behavior data for the patient;
first comparing the current behavior data to historical behavior data for the patient;
based on the first comparing, determining one or more first anomalies of the current behavior data; and
in response to the determined first anomalies, modifying the behavior of the patient.

20. The computer-readable medium of claim 19, the monitoring further comprising:
generating current biometric data for the patient;
second comparing the current biometric data to historical biometric data for the patient; and
based on the second comparing, determining one or more second anomalies of the current biometric data;
the modifying the behavior of the patient further in response to the determined second anomalies.
